(19) United States
(12) Patent Application Publication
(10) Pub. No.: US 2023/0270935 A1
(43) Pub. Date: Aug. 31, 2023

(54) CONTAINER ASSEMBLIES FOR CONTAINING AND DELIVERING MEDICAMENTS, AND METHODS OF FILLING SUCH ASSEMBLIES

(71) Applicant: West Pharmaceutical Services, Inc., Exton, PA (US)

(72) Inventors: Alex Lyness, West Chester, PA (US); Menaka Abeygunawardena, Blue Bell, PA (US); Lawton Laurence, Chester Springs, PA (US); Christian Eichhorn, Pottstown, PA (US); Samuel Molina, Lansdale, PA (US)

(21) Appl. No.: 18/086,499

(22) Filed: Dec. 21, 2022

Related U.S. Application Data
(60) Provisional application No. 63/292,408, filed on Dec. 21, 2021.

(51) Int. Cl. A61M 5/145; A61M 5/28

(52) U.S. Cl. CPC A61M 5/14566; A61M 5/283

(57) ABSTRACT

A container assembly for containing and delivering a medicament is described. The container assembly includes a container having a first end and a second end. The first end includes an opening, and a plunger assembly at least partially disposed in the container. The plunger assembly includes a main body and at least one resilient portion at least partially retained by a groove in the main body. The resilient portion forms a seal with the container. The plunger assembly forms a temperature resilient seal with the container such that the container assembly is operable to contain a medicament between the seal and the opening at: (a) room temperature, and (b) during freeze storage of the medicament. The plunger assembly is moveable relative to the container at room temperature.

### CROSS REFERENCE TO RELATED APPLICATIONS

This application claims the benefit of U.S. Provisional Patent App. No. 63/292,408, filed Dec. 21, 2021, the disclosure of which is hereby incorporated by reference herein.

### TECHNICAL FIELD

The present disclosure relates to a container assembly and pre-assembled container assemblies for containing and delivering medicaments, and to an associated method of filling such container assemblies with a medicament. The present disclosure also provides plunger assemblies for use with containers for medicaments, and resilient sealing portions for such plunger assemblies.

### BACKGROUND

Drug storage at ultralow or cryogenic temperatures is increasingly important for medicaments containing biological components, e.g., mRNA vaccines and drugs for advanced therapies such as cell or gene therapies. For example, cell therapies are generally frozen (during storage) at approximately −196° C., whereas gene therapies are generally frozen (during storage) at approximately −80° C.

One of the key challenges associated with cryogenic drug storage is maintaining container closure integrity or “CCI” (e.g., the integrity of the container seal in which the drug is stored) as the drug is cooled to its storage temperature. Moreover, in order to allow delivery of the drug to a patient at the point of use, the contents of a drug storage container must be thawed. The thawing process may comprise thawing the drug for short term storage in a refrigerator (e.g., between approximately 0° C. and 6° C.), such as at a point of care facility. The drug is generally brought to room temperature (e.g., approximately 20° C.) before being delivered to a patient.

If the drug is stored in a dedicated storage container (e.g., a vial) configured to withstand cryogenic or ultralow temperature storage conditions, the drug is generally transferred from the storage container into a delivery device (once thawed), such as a syringe, to allow delivery of the drug to a patient.

In some healthcare settings, it may be desirable for the container in which the drug is stored to also form (at least part of) the delivery device that delivers the medicament to the patient. For example, it may be advantageous for a container assembly or cartridge to be pre-filled with a medicament, frozen for storage, thawed, and then directly delivered to a patient via a delivery conduit engaged with the container.

A need therefore exists for an improved container assembly for containing and delivering medicaments, especially those stored at ultralow or cryogenic temperatures for at least a portion of their lifecycle.

### SUMMARY

In a first aspect, there is provided a container assembly for containing and optionally delivering a medicament, the container assembly comprising: a container having a first end and a second end, the first end comprising a first opening; and a plunger assembly at least partially disposed in the container, the plunger assembly comprising: a main body; and at least one resilient portion at least partially retained by a groove in the main body and forming a seal with the container; and wherein the plunger assembly is configured to: form a temperature resilient seal with the container such that the container assembly is operable to contain a medicament between the seal and the first opening at: (a) room temperature, and (b) during freeze storage of the medicament; and wherein the plunger assembly is moveable relative to the container at room temperature.

The resilient portion can comprise a resiliently deformable material that is biased towards a position in which it maintains contact with an inner wall of the container. For example, the resilient portion can comprise a ring-shaped member configured to surround the plunger main body, wherein the resilient portion is biased into a ‘rest’ position in which it contacts the inner wall of the container. By providing a resilient portion that is configured, when in situ within the container body, to be biased into contact with an inner wall of the container, examples according to the disclosure can allow the plunger assembly to retain a robust seal with the container wall across a wide temperature range. The resilient portion may optionally be deformable radially inwardly (e.g., towards the main body of the plunger assembly and into the groove). The deformability of the resilient portion can allow the resilient portion to be compressed into the groove, to facilitate sliding of the plunger assembly within the container body, when needed.

Moreover, in some configurations, by seating the resilient portion of the plunger assembly within a groove of the main body of the plunger, contraction of the plunger main body (e.g., due to freeze storage of the container assembly) may not pull the resilient portion radially inwardly away from the container wall, or may do so to a lesser extent. In this way, container assemblies according to the disclosure may provide a further improved seal under cold storage conditions.

Multiple resilient portions may be provided. The resilient portion(s) may be configured to provide an interference fit with the container.

A radially outer surface of the resilient portion(s) may extend beyond a radially outer surface of the main body of the plunger assembly. In this way, the resilient portion(s) can provide a sealing surface that contacts the inner wall of the container. The resilient portion(s) can be configured to be compressed into the grooves when the main body of the plunger assembly is placed into the container.

The container may be substantially rigid. The container may be transparent or partially transparent. Usefully, this means that the fill level of the medicament can be externally visible.

The plunger assembly may be configured, during freeze storage of the medicament, to be static relative to the container. Advantageously this can avoid accidental movement of the plunger assembly and subsequent dispelling of the medicament from the container assembly. The position of the plunger within the container body may be maintained (at least partially) by an insert configured to be placed within the container body. The interference fit and the temperature resilient seal between the resilient portion and the container wall may also maintain the plunger body in position, even during freeze storage of the medicament.

The plunger assembly and the container may be formed of materials having similar or substantially the same thermal properties. For example, the main body of the plunger assembly and the container may have similar or substantially the same thermal properties. Advantageously, in use, this means that the plunger assembly and the container can exhibit similar or substantially the same thermal expansion and contraction. This can improve the robustness of the temperature resilient seal. Alternatively, or additionally, the resilient portion(s) of the plunger may be formed of a material having similar or substantially the same thermal properties as the container, which may improve the robustness of the seal between the resilient portion(s) and the inner wall of the container.

The plunger assembly may be formed from materials suitable for use with medicaments. The main body of the plunger assembly may comprise, or consist of, an elastomeric material. The main body of the plunger assembly may be formed as a single monolithic piece, for example, a monolithic injection molded component. Alternatively, a monolithic plunger body may be formed of a rigid material, for example, it may be formed from a PTFE block.

Optionally, all or a portion of the plunger main body (especially where it is formed of an elastomeric material) may be coated with a barrier layer, such as a fluoropolymer layer. The fluoropolymer layer can be provided on at least a leading end of the plunger assembly, and may provide an inert surface for contacting a medicament container in the container assembly. The barrier layer may also extend to partially or wholly cover the generally cylindrical outer surface of the plunger main body.

The plunger assembly may be configured to be driven relative to the container by a drive element. The drive element may be a rod, for example a plunger rod. The drive element may be attachable (optionally reversibly attachable) to the plunger assembly. The drive element may in turn be configured to be driven manually, for example by the hand of a human, and/or by a drive assembly, which may be incorporated into an automatic injection device or infusion pump. The present disclosure includes automatic injection devices, infusion pumps (e.g., wearable infusion devices) and manually operated syringes comprising the container assemblies described herein.

The main body of the plunger assembly may comprise a top surface, a bottom surface and an outer surface extending therebetween. The outer surface me be substantially cylindrical and may comprise the groove. The groove may extend circumferentially around the outer surface in a direction that is substantially perpendicular to a longitudinal axis of the plunger main body. The groove may extend partially or completely around the outer surface (e.g., it may be a continuous unbroken circumferential groove, or it may be a series of discrete groove portions which together form the ‘groove’).

In at least some embodiments, the plunger assembly may comprise more than one resilient portion: for example, there may be two resilient portions, or three resilient portions. There may be at least as many grooves as resilient portions, such that each resilient portion may be seated in an associated groove. Alternatively, multiple resilient portions may be accommodated in the same groove. By providing multiple resilient portions, the resilient portion(s) can (in addition to their sealing function) stabilise the plunger assembly within the container.

In one example, the plunger assembly may comprise at least two resilient portions, wherein: one of the at least two resilient portions is configured to form the temperature resilient seal; and the other of the at least two resilient portions is configured to stabilize the plunger assembly in the container. Each of the resilient portions in this embodiment is seated within its own groove. The grooves are optionally parallel to each other and extend circumferentially around the outer cylindrical surface of the plunger body.

The resilient portion(s) may each have substantially the same configuration, or they may have different configurations to suit their primary purpose. For example, a first resilient portion (e.g., a resilient portion arranged towards a leading edge of the plunger main body) may be coated with an inert barrier layer that is configured to prevent or minimise interaction between the plunger and the medicament container within the container. A second resilient portion (e.g., a resilient portion arranged towards a trailing edge of the plunger main body) may not be coated, since it is not generally in contact with the medicament contained within the container. It will of course be appreciated that both of the resilient portions can be coated with a barrier layer to e.g., facilitate sliding of the plunger assembly within the container body.

The resilient portion(s) can comprise an encapsulated O-ring. The encapsulated O-ring can comprise a core and a jacket. The jacket may partially or wholly encapsulate the core.

The core may comprise or be entirely formed from a resilient material, such as: a spring, and/or a polymer. The polymer may be solid or hollow. The polymer may be silicone, for example. The spring may be a continuous contact spring, a cantilever spring, a helical-wound spring, and/or an elliptical coil spring. The spring may be made of a metallic material. The jacket may be formed from a fluoropolymer material, such as: fluorinated ethylene propylene (FEP) and/or perfluoroalkoxy alkane (PFA). Advantageously, the core is configured to extend the operating limits of the jacket, for example, such that the temperature resilient seal is maintained across a broader temperature range, and especially at lower temperatures.

The resilient portion may be a spring energised seal. The spring energised seal can comprise a jacket and a core, similar to an encapsulated O-ring, or it may comprise a spring and a sealing ring. The sealing ring is configured to provide a sealing surface for contacting the inner surface of the container, whilst the spring is configured to bias the sealing ring into contact with the inner surface. The sealing ring (and/or the jacket) may comprise a fluoropolymer material, such as one or more fluoropolymer layers.

The container assembly may be configured to receive the medicament via the first opening in the first end. The container assembly may also be configured to deliver the medicament via the first opening in the first end. Accordingly, the plunger assembly may be configured to be driven (at least) in a direction from the second end towards the first end.

The container assembly may additionally comprise a stopper assembly configured to seal the first opening of the container. The stopper assembly may be at least partially disposed in the first opening of the container. The stopper assembly may optionally be configured to form a further temperature resilient seal with the container such that the seal formed by the stopper assembly is operable for use at room temperature and during freeze storage of the medicament.

The stopper assembly comprises a stopper having a generally cylindrical outer surface and a top surface, comprising a flange that extends beyond in a radial direction, beyond the generally cylindrical outer surface. When disposed in the first opening, the generally cylindrical outer surface of the stopper may face the inner wall of the container. The flange is configured to abut a rim of the container. The stopper is configured to form a seal with the container to seal the first opening. The generally cylindrical body of the stopper assembly may be substantially hollow, such that the top surface of the stopper acts as a septum through which a needle can be inserted to establish fluid communication between the internal volume of the container and a delivery conduit.

The stopper assembly may further comprise at least one resilient sealing portion. The stopper may comprise at least one groove. The resilient portion may be configured to be accepted by the at least one groove. That is, the resilient portion may be configured to be at least partially arranged or seated in the groove. The resilient sealing portion may extend beyond a surface of the stopper to provide a sealing surface for contacting the container. The resilient sealing portion may be configured to provide an interference fit with the container. The resilient sealing portion may be configured to form the further temperature resistant seal. Advantageously, the arrangement of the resilient sealing portion in the stopper creates a robust seal between the stopper assembly and the container.

In one example, the at least one resilient sealing portion is configured to provide the interference fit with the inner wall of the container. That is, to provide a radial seal with the container. In such a configuration, the groove is formed in the generally cylindrical outer surface of the stopper, and the resilient sealing portion is seated therein.

Additionally or alternatively, the at least one resilient portion and/or the stopper assembly is configured to form a seal with a rim of the container (e.g., a rim surrounding the first opening). In such a configuration, the resilient sealing portion is seated within a groove that extends around a flange of the stopper, such that it may be brought into abutment with the rim of the container. In other words, the resilient sealing portion may be configured to provide an axial seal with the container.

The resilient sealing portion(s) associated with the stopper described above can take the form of an (encapsulated) O-ring or a spring energised seal, having a construction similar to that described above in relation to the plunger assembly.

The stopper assembly may additionally comprise a first end cap configured to hold the stopper in place over the opening of the container. The end cap can be configured to compress the stopper against a wall or rim of the container. The end cap may be a cap (e.g., a snap-fit or threaded cap) or a crimped seal, for example an aluminium crimped seal.

The container assembly may additionally comprise an insert at least partially disposed in the container and configured to maintain a minimum distance between the plunger assembly and the second end of the container, which may comprise a second opening.

The container assembly may additionally comprise a second end cap. The second end cap may be configured to close the second opening at the second end of the container, and optionally to retain the insert (if present) in the container. In one example, the second end cap is configured for reversible attachment to the container. The second end cap may also be irreversibly attached to the container or frangibly engaged with the container, for example, such that reuse of the container is prevented. The second end cap may be configured to provide a stable base for the container.

In some embodiments, and in particular those configured for open filling of the container, the second end cap is configured to be secured in place over the second end of the container to close the second opening of the container prior to filling. The container can then be filled via the first opening, before the first opening is sealed with the stopper to provide a sealed medicament container. In such a configuration, the second end cap seals the second open end of the container.

In other embodiments, the container assembly can be configured for closed filling. In such embodiments, the second end cap can take the form of a vented end cap comprising at least one opening. The vented end cap is secured over the second opening of the container (at the second end). The openings allow the flow of air out of the container via the at least one opening. Optionally, a filter may be provided that extends across the opening(s) to prevent the ingress of material into the container. The openings can allow air to escape from the container body as the plunger assembly is driven from the first end of the container towards the second end of the container during a filling step.

A check valve can be provided at the first opening of the container. The check valve can be configured as a one-way valve. The one-way valve can be configured to allow the flow of fluid into the container via the first opening, but to prevent the flow of fluid out of the container unless the valve is opened, for example using a valve adaptor. Optionally, the valve may be co-molded with the container.

A valve cover can further be provided to close the first end of the container and cover the check valve. The valve cover may be reversibly attachable to the container. The valve cover may also be frangibly connected to the container, such that the container cannot be reused.

In a second aspect, there is provided a first pre-assembled container assembly, the first pre-assembled container assembly comprising: the container assembly of the first aspect; an insert at least partially disposed in the container and configured to maintain a minimum distance between the plunger assembly and the second end of the container; and a second end cap attached to or engaged with the container and configured to retain the insert in the container. The insert is configured to maintain a minimum distance between the plunger assembly and the second end cap. The pre-assembled container of the second aspect can include any of the features described above in connection with the first aspect.

The first pre-assembled container assembly may be sterilised. The first pre-assembled container assembly may be wrapped, for example individually.

In a third aspect, there is provided a second pre-assembled container assembly, the second pre-assembled container assembly comprising: the container assembly of the first aspect, wherein the second end of the container comprises a second opening; a vented end cap attached to or engaged with the container at or near the second end of the container and comprising one or more openings configured to allow air to pass into and out of the container through the second opening; and a check valve at least partially disposed in the container at or near the first end of the container and configured to allow fluid flow out of the container via the first opening in the first end. The pre-assembled container of the third aspect can include any of the features described above in connection with the first aspect.

The second pre-assembled container assembly may be sterilised. The second pre-assembled container assembly may be wrapped, for example individually.

The second pre-assembled container assembly may be supplied with a valve adaptor. The valve adaptor may be configured for engagement with the check valve and configured to allow fluid flow through the check valve against the one-way flow direction for which the check valve is configured.

In at least some embodiments, the check valve may be configured to allow fluid flow into the container, and to prevent fluid flow out of the container unless the valve adaptor is engaged with the check valve.

The valve adaptor may be supplied engaged with the check valve, or it may be supplied as part of a kit including the valve adaptor and a container assembly comprising a check valve as described above.

In a fourth aspect, there is provided a plunger assembly, as described with reference to any of the aspects set out above, in isolation from the container assembly in which the plunger assembly is disposed in the first to third aspects. In other words, there is provided a plunger assembly comprising: a main body and at least one resilient portion at least partially retained by a groove in the main body and configured to form a seal with a container. The resilient portion can comprise a core and a jacket, the jacket at least partially encapsulating the core. The core can comprise a spring and/or a polymer. The polymer may be silicone. The jacket can comprise a fluoropolymer material.

The at least one resilient portion can comprise a spring energised seal, and wherein the spring energised seal comprises a spring and a sealing ring. The spring can be a continuous contact spring, a cantilever spring, a helical-wound spring, or an elliptical coil spring. The sealing ring can comprise a fluoropolymer material.

In a fifth aspect, there is provided a stopper assembly configured to seal an opening of a container, the stopper assembly comprising: a stopper; and at least one resilient sealing portion at least partially retained by a groove in the stopper. One or more features of this fifth aspect may be as described in relation to any other aspect. For example, the resilient sealing portion of the stopper assembly may be the same or substantially similar to the resilient portion described above with reference to the plunger assembly.

In a sixth aspect, there is provided a resilient portion as described with reference to any of the aspects set out above, in isolation from the container assembly and the plunger main body (or stopper) about which the resilient portion extends. In other words, there is provided a resilient portion comprising an encapsulated O-ring or a spring energised seal. The resilient portion can comprise a core and a jacket, the jacket at least partially encapsulating the core. The core can comprise a spring and/or a polymer. The polymer may be silicone. The jacket can comprise a fluoropolymer material.

The at least one resilient portion can comprise a spring energised seal, comprising a spring and an optional sealing ring. The spring energised seal may comprise a jacket that encapsulates the spring, and optionally the sealing ring. The spring can be a continuous contact spring, a cantilever spring, a helical-wound spring, or an elliptical coil spring. The sealing ring and/or the jacket can comprise a fluoropolymer material.

In a seventh aspect, there is provided a method of filling a container assembly according to the disclosure. The container assembly may be the container assembly of any other aspect. The method comprises the step of filling the container assembly with a medicament via the first opening in the first end.

The method can optionally include filing the container through an open or unoccluded first opening and subsequently closing the first opening of the container assembly, for example with a stopper. Alternatively, the container may be filled via a one-way check valve disposed in the first opening.

The method may comprise, after filling the container assembly with the medicament, freezing the medicament contained in the container assembly. The method may comprise, after freezing the medicament contained in the container assembly, freeze storing the medicament contained in the container assembly. The method may comprise, after freezing the medicament contained in the container assembly, thawing the medicament contained in the container assembly.

The method may comprise, after thawing the medicament contained in the container assembly, delivering the medicament from the container assembly, optionally via the first opening. The medicament may be delivered to patient, or to another container. The step of delivering the medicament may comprise driving the plunger assembly towards the first end.

In an eighth aspect, there is provided a method of manufacturing a container assembly according to any of the aspects described above. The method includes the steps of providing a container having a first end and a second end; providing a plunger assembly disposed at least partially in the container such that a seal is formed between the plunger assembly and an inner wall of the container. The seal may be formed between the inner wall of the container and a resilient portion, such as a resilient portion described in connection with any of the aspects above.

The method optionally comprises closing the first end of the container. This can be done, for example, with a one-way check valve. The one-way check valve may be integrally formed with the container, for example, it may be co-molded. Alternatively, the one-way check valve may be secured in place within the first opening of the container after formation of the container body. A stopper may also be used to close the first end of the container. The second end of the container can be closed with, for example, an end cap.

The filling steps described above in connection with the seventh aspect may be incorporated into the method of manufacturing of the eighth aspect. In an ‘open-fill’ method, the container is filled through an open or unoccluded first end of the container. The medicament is introduced into the space between the plunger assembly and the open first end. In a closed fill method, the container is filled through a check-valve disposed at the first end of the container. The medicament is introduced via the check valve and drives the plunger assembly from an initial position adjacent the check-valve, towards the second end of the container.

For each of the above aspects, room temperature may be any temperature in the range suitable for the delivery of the medicament to a patient. For example, room temperature may be any temperature in the range of around 0° C. to around 50° C. Room temperature may be around 15° C. to around 40° C. Room temperature may be around 20° C. to around 30° C. Room temperature may be around 20° C. to around 25° C. Room temperature may be around 20° C. to around 22° C. For example, room temperature may be around 20° C., or at around 22° C.

Freeze storage of the medicament may be carried out at any temperature suitable for freeze storing the medicament, for example any temperature below 0° C. In at least some examples, freeze storing of a medicament is carried out at cryogenic and/or at ultralow temperatures. Freeze storage may be carried out at any temperature in the range of around 0° C. to around −200° C., for example, at any temperature below −20° C., at any temperature below −50° C., at any temperature below −60° C., for example, at any temperature at or below around −80° C. In some embodiments, freeze storage is carried out at a temperature of around −196° C.

The temperature resilient seal may be operable (i.e., maintain container closure integrity or “CCI”) at temperatures suitable for gene and/or cell therapy. For example, the seal may be operable at any temperature in the range of around 0° C. to around −200° C., for example, at any temperature below −20° C., at any temperature below −50° C., at any temperature below −60° C., for example, at a temperature at or below around −80° C. In at least some configurations, the temperature resilient seal is operable at a temperature of around −196° C.

Further embodiments and advantages will become apparent from the following detailed description and the appended claims, as well as the drawings.

### BRIEF DESCRIPTION OF THE DRAWINGS

The invention will now be described in more detail with reference to a number of non-limiting, exemplary embodiments shown in the following drawings, in which:

FIG. 1 shows an exploded view of a container assembly according to a first embodiment;

FIG. 2a shows a side view of a first plunger assembly suitable for use in the container assembly of FIG. 1;

FIG. 2b shows a cross-sectional view of the plunger assembly of FIG. 2a;

FIG. 3a shows a top view of a resilient component suitable for use with the plunger assembly of FIGS. 2a and 2b;

FIG. 3b shows a cross-sectional view of a portion of the resilient component of FIG. 3a;

FIG. 4 shows an alternative resilient component;

FIG. 5 shows an exploded view of a container assembly suitable for open filling, which comprises an end cap and a stopper;

FIG. 6a shows a first configuration for the end cap and stopper of FIG. 5;

FIG. 6b shows a second configuration for the end cap and stopper of FIG. 5;

FIG. 6c shows a third configuration for the end cap and stopper of FIG. 5 in a disassembled state;

FIG. 6d shows the third configuration for the end cap and stopper of FIG. 5 in an assembled state;

FIG. 7 shows a seal configuration for a stopper comprising a resilient component according to a first configuration;

FIG. 8 shows a seal configuration for a stopper comprising a resilient component configuration according to a second configuration;

FIG. 9 shows the container assembly of FIG. 5 during a filling step;

FIG. 10 shows the container assembly of FIG. 5, after the filling step of FIG. 9 and during placement of the end cap and stopper;

FIG. 11 shows the container assembly of FIG. 5 being prepared for use;

FIG. 12 shows the container assembly of FIG. 5 as part of a drug delivery system;

FIG. 13 shows an exploded view of a container assembly suitable for closed filling, which comprises a vented end cap and a check valve;

FIG. 14a shows a first arrangement for the vented end cap of FIG. 13, in which an insert according to first configuration is provided, before the container is filled;

FIG. 14b shows the vented end cap of FIG. 14a, after the container is filled;

FIG. 14c shows an arrangement for the vented end cap of FIG. 13, in which an insert according to a second configuration is provided;

FIG. 14d shows an arrangement for the vented end cap of FIG. 13, in which an insert according to a third configuration is provided;

FIG. 14e shows an arrangement for the vented end cap of FIG. 13, in a disassembled state, in which an insert according to a fourth configuration is provided;

FIG. 14f shows the arrangement of FIG. 14e in an assembled state;

FIG. 15 shows the container assembly of FIG. 13 after a filling step;

FIG. 16 shows the container assembly of FIG. 13 being prepared for use;

FIG. 17 shows a flowchart displaying the steps of a first method according to the disclosure;

FIG. 18 shows a flowchart displaying the step of a second method according to the disclosure.

Like reference numerals are used for like components throughout the drawings.

### DETAILED DESCRIPTION

The present disclosure is directed generally to a plunger for a container assembly, a container assembly comprising a plunger, and associated methods for manufacturing and using container assemblies according to the disclosure. The plunger assembly and container assemblies incorporating it are generally well suited to maintaining a seal at very low temperatures (e.g., medicaments stored at ultralow or cryogenic temperatures), whilst still being manually slidable within a container body at room temperatures.

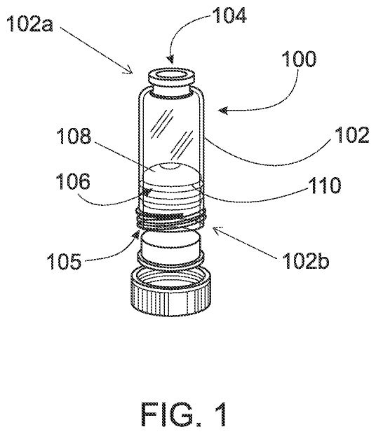

FIG. 1 shows a container assembly 100 for containing and delivering a medicament. The container assembly 100 comprises a container 102 that extends from a first (distal) end 102a to a second (proximal) end 102b. The first end 102a of the container assembly comprises a first opening 104 which provides fluid communication between an internal volume of the container 102 and an exterior of the container 102. The second end 102b of the container 102 comprises a second opening 105.

The container can be made of a glass or polymeric material. In at least one configuration, the container 102 comprises a clear plastic material, such as cyclic olefin polymer (COP), cyclic olefin copolymer (COC) and/or polypropylene (PP). Container bodies suitable for use in assemblies according to the present disclosure may be formed by injection moulding or blow moulding.

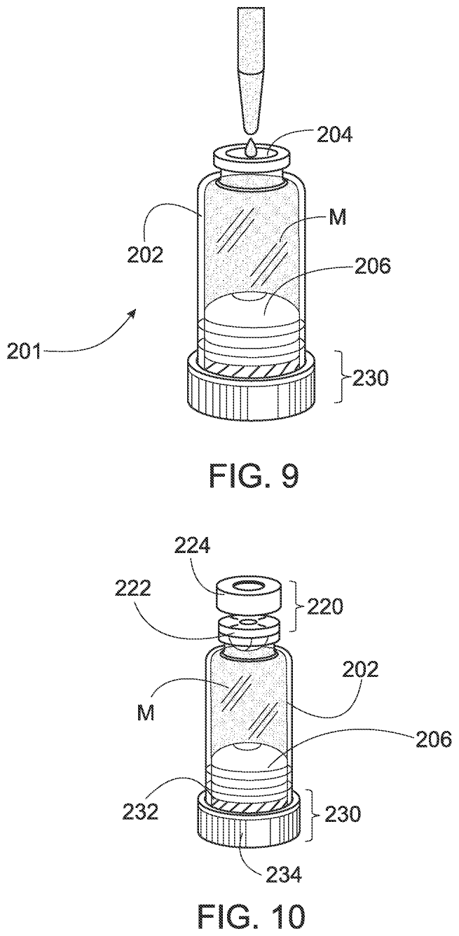

The container assembly 100 further comprises a plunger assembly 106. The plunger assembly 106 is configured to be seated at least partially within the internal volume of the container 102 and to form a seal with an inner surface of the generally cylindrical container body 102. The plunger assembly 106 is also configured to be moved relative to the container 102 by a plunger rod (shown in FIG. 9) to discharge a dose of medicament from the internal volume of the container 102.

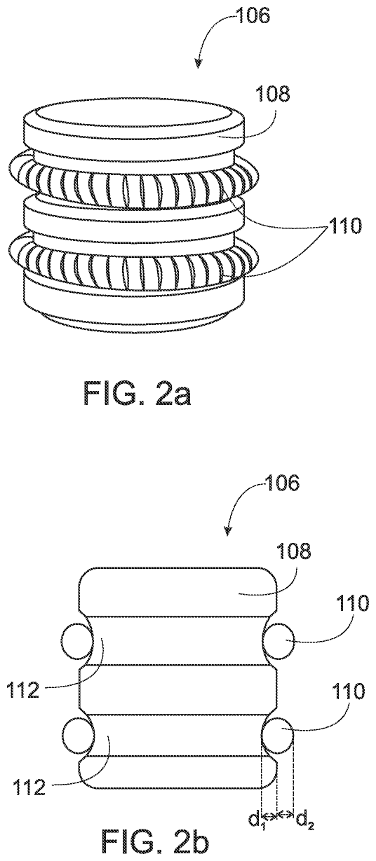

Referring now to FIGS. 2a and 2b, the plunger assembly 106 comprises a main body 108 and at least one resilient portion 110 retained by a groove (shown in FIG. 2b) in the main body 108. More specifically, the main body 108 can comprise a top surface, a bottom surface, and a generally cylindrical outer surface extending between the top and bottom surfaces. In the configuration shown in FIGS. 2a and 2b, the main body 108 comprises two grooves 112, each configured to receive a resilient portion 110. The groove(s) 112 are formed in the outer surface of the main body 108. The grooves 112 extend circumferentially around the outer surface of the main body 108, completely encircling the body 108, and have a uniform depth about the circumference. However, it will be appreciated that the grooves 112 need not completely encircle the body 108, nor have a uniform depth about their circumference to receive a resilient portion. It will be appreciated that the groove geometry can be selected based on the design of the resilient portion, the seal type, or container closure integrity requirements. For example, the groove can comprise a cylindrical groove, a dovetail groove, a half dovetail groove, or a triangular crush groove.

The main body 108 may be formed of an elastomeric material, for example: rubbers, halogenated butyl rubbers, silicon rubbers, isoprene rubbers, and Styrene butadiene rubbers. Alternatively, the main body 108 can comprise a rigid polymer, such as a fluoropolymer (including one or more of PTFE, PCTFE, FEP) or a clear plastic material (including one or more of COP, COC, PP). In a preferred embodiment, at least one of the top surface or bottom surface of the main body is laminated with an inert film, most preferably the surface of the main body 108 that will contact the medicament stored within the container. Examples of inert films include, but are not limited to, fluorinated polymers, such as ETFE, PTFE, FEP and monolayer polyolefin films such as COP, COC, and PE. The main body 108 may be a monolithic component, i.e., formed as a single piece. The main body 108 can be an injection-moulded, compression moulded, or machined.

The main body 108 of the plunger assembly 106 can be made from a material that has similar or equivalent thermal expansion properties as the container 102 such as plastic materials (including one or more of COP, COC, PP) to ensure consistent expansion and contraction between the plunger assembly 106 and the container 102.

Referring now to FIG. 2b, the groove 112 can have a diameter that is less than a diameter of an adjacent portion of the outer surface of the main body 108. In other words, the deepest portion of the groove 112 can be recessed from the outer surface of the main body 108 by distance d1.

The distance d1 can be less than a thickness (measured in a radial direction) of the resilient portion 110. In this way, the groove 112 can be configured to partially receive the resilient portion 110 so that the outer surface of the resilient portion 110 extends beyond at least a portion of the outer surface of the main body 108 by a distance d2. This ensures that the resilient portion 110 provides a sealing surface for contacting an inner surface of the container 102. In this way, the resilient portion 110 can be configured to provide an interference fit with the container 102. In at least some configurations, it is preferred that the sealing surface(s) provided by the resilient portion(s) have an outer diameter that is approximately equal to an outer diameter of a leading portion of the plunger. For example, the main body of the plunger may have a shape similar to that described in U.S. Pat. No. 10,258,744. In such a configuration, the resilient portion(s) 110 take the place of (or are provided in addition to) the ribs of the plunger described in U.S. Pat. No. 10,258,744.

Although the configuration shown in FIGS. 2a and 2b comprises two circumferential grooves 112 and two resilient portions 110, it will be appreciated that a single resilient portion can be provided, or that three of more resilient portions 110 may be present.

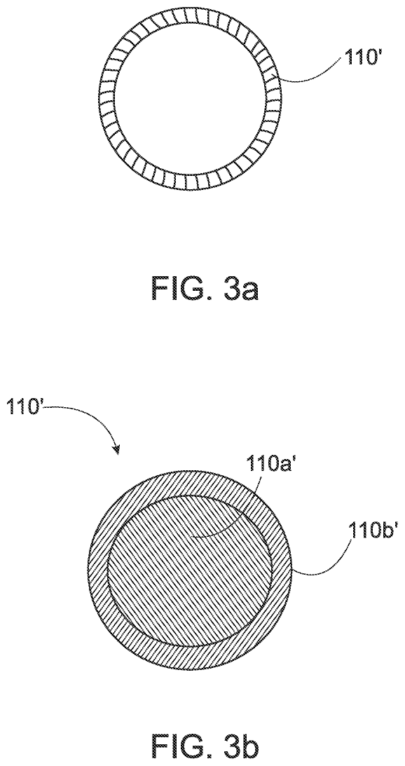

As shown in FIG. 3a, the resilient portion can take the form of an O-ring 110′ configured to sit within the circumferential groove 112 formed in the outer surface of the main body 108. The O-ring can be formed of a resiliently deformable material, such that it can be compressed against the inner surface of the container 102 to form an interference fit.

Referring to FIG. 3b, the O-ring 110′ can be an encapsulated O-ring consisting of two parts: a core material 110a′ and a jacket 110b′ that at least partially encapsulates the core material. The core 110a′ can be a hollow stainless steel metal spring or a hollow or solid polymer, such as silicone and Viton. The core material is resiliently deformable such that it tends to resist compression in at least a radially inward direction. In this manner, the core can be configured to bias an outer sealing surface of the O-ring 110′ into sealing contact with the inner surface of the container 102 when the plunger assembly 106 is disposed within the container 102. The core 110a′ may be monolithic (formed as a single piece) or may comprise multiple connected resilient components.

The jacket 110b′, which partially or wholly encapsulates the core 110a′, can comprise an inert material (compatible with drug products) and/or one that can provide low frictional engagement with the inner surface of the container body 102. The jacket 110b′ may comprise a fluoropolymer material such as fluorinated ethylene propylene (FEP) and/or perfluoroalkoxy alkane (PFA).

The jacket 110b′ can act to provide a smooth, continuous sealing surface for contacting the inner surface of the container 102. It will be appreciated that this can allow the core 110a′ to be formed of a material and/or structure that would otherwise be unsuitable for sealing, e.g., a coiled spring.

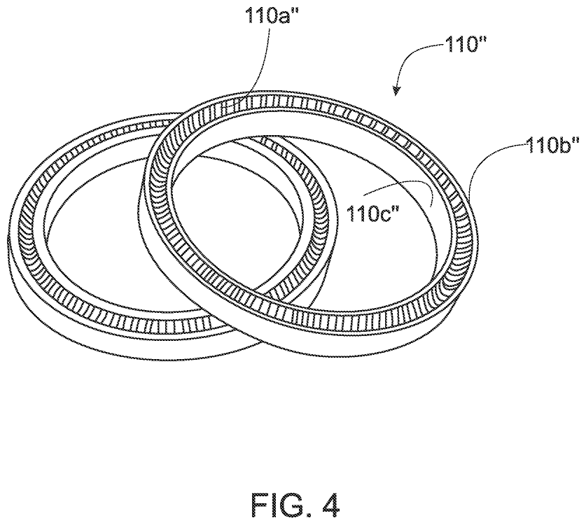

Referring now to FIG. 4, the resilient portion may also take the form of a spring energised seal 110″. The spring energised seal 110″ can comprise a spring 110a″ and an outer sealing ring 110b″. The outer sealing ring 110b″ is configured to be biased in a radially outward direction by the spring 110a″. In the embodiment shown in FIG. 4, the spring 110a″ is disposed between an outer sealing ring 110b″ and an inner ring 110c″. However, it will be appreciated that the inner ring 110c″ may be omitted in some configurations.

The spring 110a″ can take the form of a continuous contact spring, a cantilever spring, a helical spring, or an elliptical coil. Alternatively, the spring 110a″ can be formed of a hollow or solid polymeric ring. A single spring 110a″ may be used to bias the sealing ring 110b″ in a radially outward direction, or a plurality of springs may be used to perform this function. The outer sealing ring 110b″ is advantageously formed of a material that is compatible with drug products and one that can provide low frictional engagement with an inner surface of the container 102. A suitable material for the outer sealing ring 110b″ may be elastomeric materials such as butyl rubbers, halogenated butyl rubbers or fluoropolymers such as FEP and PFA. The outer sealing ring may be further coated with an inert material, at least on a portion of its surface.

The configuration of the resilient portion(s) 110, 110′, 110″ described above is such that a temperature resilient seal may be formed between the plunger assembly and the container 102 so that the container assembly is operable to contain a medicament between the seal and the opening at: (a) room temperature (e.g. approximately 20° C.), and (b) during freeze storage of the medicament, with the plunger assembly still being movable relative to the container at room temperature.

Referring again to FIGS. 2a and 2b, the main body 108 of the plunger assembly 106 is shown to comprise two grooves 112, each configured to receive a resilient portion 110. In the configuration shown in FIGS. 2a and 2b, each of the grooves 112 and each of the resilient portions 110 are the same. However, in at least one embodiment, two resilient portions 110 may be provided, in which one of the resilient portions 110 is configured to form a temperature resilient seal, whereas the other resilient portion 110 is configured to stabilize the plunger assembly 106 within the container 102. In such cases, the configuration of the resilient portions 110 may be the same. For example, the resilient portion closest to the leading end of the plunger may act to form a seal that prevents egress of medicament past the resilient portion, whereas the resilient portion closest to the trailing end of the plunger can act to stabilise the plunger within the syringe body, with each of the resilient portions being substantially the same in terms of materials and dimensions. However, the skilled person will appreciate that the dimensions and/or materials used to form the leading and trailing resilient portions may be chosen to improve the performance of their respective functions. For example, the leading resilient portion may comprise an inert film layer on at least a portion of its surface, since it is intended to come into contact with a medicament contained within the plunger. The trailing resilient portion may be substantially free of an inert barrier layer. Alternatively or additionally, the thickness and/or outer diameter of the leading resilient portion may be greater or smaller than the thickness and/or outer diameter of the trailing resilient portion to provide improved sealing of the leading resilient portion compared to the trailing resilient portion.

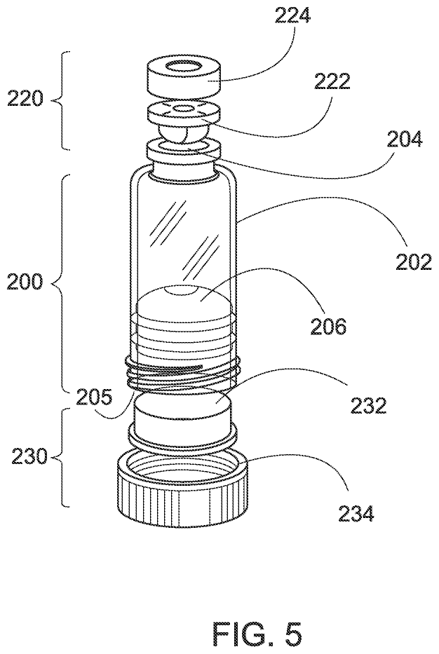

Referring now to FIG. 5, an open fill container system according to the disclosure will now be described in more detail. As shown in FIG. 5, the container system comprises a container assembly 200, which is similar in structure to the container assembly 100 described above. The container assembly 200 comprises a container 202 and a plunger assembly 206 disposed therein. The plunger assembly 206 has a similar construction to the plunger assembly 106 described above. The container 202 has a first opening 204 at its first end, and a second opening 205 at its second end.

The open fill container system further comprises a stopper assembly 220 configured to close the open (distal) end of the container 202, and an insert assembly 230 configured to maintain a minimum distance between the plunger assembly 206 and the second (proximal) end of the container 202. By providing an insert that limits the position of the plunger assembly, it is possible to improve control over placement of the plunger assembly with the container body, thus improving the accuracy with which the interior fill volume of the container can be defined.

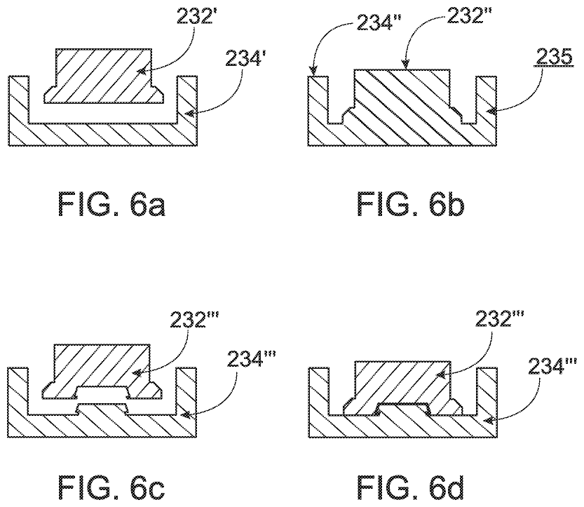

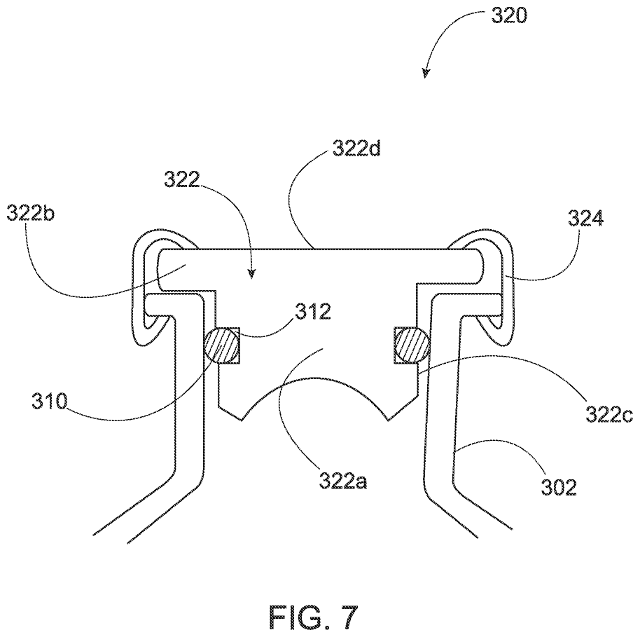

As shown in FIG. 5, the stopper assembly 220 comprises a stopper 222, at least partially disposed within the opening of the container 202 and a first end cap 224 configured to maintain the stopper 222 in place within the opening. The stopper 222 may be an elastomeric stopper configured to form a seal against a rim surrounding the opening of the container 202. In at least one configuration, the stopper assembly 220 can comprise a resilient component similar to the resilient portions 110, 110′, 110″ described above. Further detail on such configurations is explained with reference to FIGS. 6 and 7 below. The first end cap 224 can comprise a crimped seal (e.g., formed of a ductile metal material, such as aluminium) configured to compress the stopper 222 against the rim of the container 202. Alternatively, the first end cap 224 can comprise a snap fit cap, a screw threaded cap, or other cap arrangement configured to hold the stopper 222 in place. Moreover, frangible connections between the end cap and the container may also be used to help ensure that the container is used only once.

The end cap may be formed from one or more rigid polymeric materials, such as polyethylene (PE), polypropylene (PP), polyvinyl chloride (PVC), polystyrene (PS), polyethylene terephthalate (PET) or acrylonitrile butadiene styrene (ABS). Advantageously, the material chosen for the end cap is compatible with cryopreservation media such as dimethyl sulfoxide (DMSO).

The insert assembly 230 comprises an insert 232 at least partially disposed in the container 202 and configured to maintain a minimum distance between the plunger assembly 206 and the second (proximal) end of the container 202. The insert 232 is in turn held within the container body 202 by a second end cap 234. The second end cap 234 may be threadedly engaged with the container 202 by way of complementary screw threads provided on the container 202 and the second end cap 234. However, the second end cap 234 may also be snap fit over the container body, or otherwise engaged with the container body to maintain the insert 232 within the interior volume of the container 202, between the plunger assembly 206 and the second (proximal) end of the container. In this way, the insert 232 can act as a spacer between the second end cap 234 and the plunger 206, to ensure that a minimum distance between the plunger assembly 206 and the second (proximal) end of the container 202. Moreover, frangible connections between the end cap and the container may also be used to help ensure that the container is used only once.

As will now be described with reference to FIGS. 6a-6d, the insert assembly 230, which comprises the end cap 234 and the insert 232, can take different forms.

For example, as shown in FIG. 6a, the insert assembly can comprise an end cap 234′ and a separate insert 232′. In this embodiment, the end cap 234′ and the insert 232′ are not coupled or connected to each other, and instead the insert 232′, acts as a spacer when confined between the end cap and the plunger, thereby defining a minimum distance between the open end of the container 202 and the plunger. When the end cap 234′ is removed, the insert 232′ can be tipped or pulled out of the open end of the container 202, leaving the plunger 206 accessible for use. It will be appreciated that having separate end cap 234′ and insert 232′ components allows the size of the insert 232′ to be varied independently of the end cap 234′ (to vary the minimum distance between the open end of the container and the plunger).

In a second configuration shown in FIG. 6b, the insert assembly can comprise an end cap and an insert combined as a monolithic body 235, or otherwise permanently secured to each other (e.g., with adhesive). In this configuration, the monolithic body 235 comprises an end cap portion 234″, which is configured to engage the container 202, whilst the insert portion 232″ is configured to extend from the end cap portion 234″ within the inner volume of the container 202 to define a minimum distance between the open end of the container 202 and the plunger. The insert portion 232″ can comprise an extension (e.g., a generally cylindrical extension) or a plurality of extensions (e.g., a plurality of legs or posts) extending from the end cap portion 234″ towards the plunger 206. The extension(s) are configured to provide an abutment surface against which the plunger 206 bears to prevent further advancement of the plunger 506 towards the open end of the container 202. In such a configuration, when the end cap portion 234″ is removed from the container 202, the insert portion 232″, which is fixedly attached thereto, is also removed from open end of the container 202, leaving the plunger 206 accessible for use.

In a third configuration, the insert assembly can comprise an end cap and insert configured for snap-fit engagement with each other (or another form of selective engagement). FIG. 6c shows the arrangement in a disassembled state, in which the insert and the end cap are disconnected from each other. FIG. 6d shows the arrangement in an assembled state, in which the end cap and the insert are connected.

For example, as shown in FIGS. 6c and 6d, the insert assembly can comprise an end cap 234′″ and a separate insert 232′″ configured to engage the end cap 234′″. The engagement between the end cap 234′″ and the insert 232′″ can be snap-fit, interference fit, or a threaded engagement. In this configuration, the end cap 234′″ is configured to engage the container 202, whilst the insert 232′″ is configured to define a minimum distance between the open end of the container 202 and the plunger. In such an arrangement, because the end cap 234′″ and the inert 232′″ are connected to each other (e.g., by snap fit engagement) removal of the end cap 234′″ from the container 202 also removes the insert 232′″ from the open end of the container 202, leaving the plunger 206 accessible for use. It will be appreciated that an advantage of this connected but modular construction of the insert 232′″ and the end cap 234′″ means that the size of the insert 232′″ can be varied (to vary the minimum distance between the plunger and the open end of the container) independently of the end cap 234′″, but removal of the end cap 234′″ can still remove the insert 232′″ from the open end of the container.

The insert assembly (or at least one portion thereof) may be formed from one or more rigid polymeric materials, such as polyethylene (PE), polypropylene (PP), polyvinyl chloride (PVC), polystyrene (PS), polyethylene terephthalate (PET) or acrylonitrile butadiene styrene (ABS). Advantageously, the material chosen for the insert assembly is compatible with cryopreservation media such as dimethyl sulfoxide (DMSO).

Referring now to FIG. 7, a first configuration of a stopper assembly comprising a resilient portion similar to the resilient portions 110, 110′, 110″ will now be described.

As shown in FIG. 7, a stopper assembly 320 can comprise a stopper 322 configured to sit at least partially within an opening of a container 302. The stopper 322 is maintained in place with a first end cap 324. The first end cap 324 can be a crimped seal, a snap-fit cap, or other cap assembly configured to hold the stopper 322 in place within a neck of a container.

The stopper 322 comprises a generally cylindrical body 322a, configured to sit within a neck of container 302. The stopper 322 also comprises a flange 322b, extending in a radial direction from the generally cylindrical body 322a, which abuts a rim surrounding the opening of the container 302. The cylindrical body 322a comprises an outer surface 322c. The generally cylindrical body 322a can be closed at the first end (the end comprising the flange 322b), and open at the opposing second end. In this way, the stopper 322 can comprise a septum portion 322d through which access to the interior volume of the container 302 may be accessed e.g., by a needle piercing the septum 322d.

The stopper 322 comprises a groove 312, which extends around the outer surface 322c of the generally cylindrical portion 322a. The groove 312 at least partially receives a resilient sealing portion 310. As shown in FIG. 7, the resilient sealing portion 310 is seated within the groove 312.

The sealing portion 310 can take the form of an O-ring, similar to the O-ring 110′ described above. The O-ring 310 can have a thickness (in the radial direction) that is greater than or equal to the depth of the groove, such that an outer sealing surface of the O-ring extends radially to a distance that is greater than or equal to the outer surface of the generally cylindrical body 322a. In this manner, the O-ring can provide an interference fit with an inner surface of the container 302 to maintain a seal between the stopper 322 and the container 302.

The O-ring 310 can be an encapsulated O-ring consisting of two parts: a core material and a jacket that at least partially encapsulates the core material. The construction of the O-ring 310 can be the same or similar as the construction shown in cross-section in FIG. 3b. The core can be a hollow stainless steel metal spring or a hollow or solid polymer, such as silicone. The core material is resiliently deformable such that it tends to resist compression in at least a radially inward direction. In this manner, the core can be configured to bias an outer sealing surface of the O-ring 310 into sealing contact with the inner surface of the container 302 when the stopper 322 is disposed within the neck of the container 102.

The core may be monolithic (formed as a single piece) or may comprise multiple connected resilient components.

The jacket, which partially or wholly encapsulates the core, can comprise an inert material (compatible with drug products) and/or one that can provide low frictional engagement with the inner surface of the container body 302. The jacket may comprise a fluoropolymer material such as fluorinated ethylene propylene (FEP) and/or perfluoroalkoxy alkane (PFA).

The jacket can act to provide a smooth, continuous sealing surface for contacting the inner surface of the container 302. It will be appreciated that this can allow the core to be formed of a material and/or structure that would otherwise be unsuitable for sealing, e.g., a coiled spring.

The resilient sealing portion 310 may also take the form of a spring energised seal, for example, constructed according to the configuration described above with reference to FIG. 4. The spring energised seal can comprise a spring and an outer sealing ring. The outer sealing ring is configured to be biased in a radially outward direction by the spring. The spring can be disposed between an outer sealing ring and an inner ring. Alternatively, the inner ring may be omitted in some configurations.

In this configuration, the spring can take the form of a continuous contact spring, a cantilever spring, a helical spring, or an elliptical coil. A single spring may be used to bias the sealing ring in a radially outward direction, or a plurality of springs may be used to perform this function. The outer sealing ring is advantageously formed of a material that is compatible with drug products and one that can provide low frictional engagement with an inner surface of the container. A suitable material for the outer sealing ring may be elastomeric materials such as butyl rubbers, halogenated butyl rubbers or fluoropolymers such as FEP and PFA.

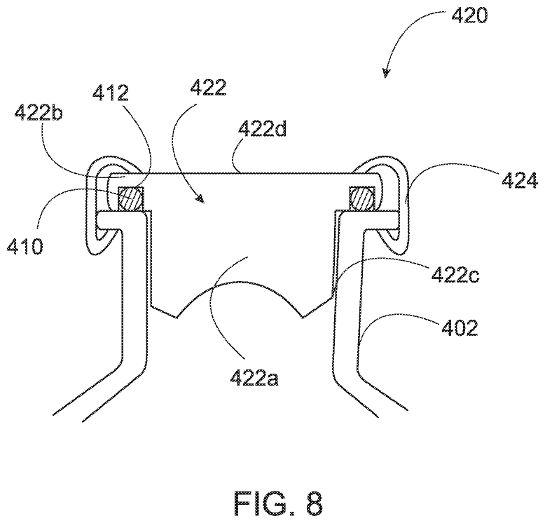

FIG. 8 shows an alternative stopper assembly 420, wherein a resilient sealing portion 410 is seated within a groove in flange 422b.

As shown in FIG. 8, the stopper 422 comprises a generally cylindrical body 422a configured to sit within a neck of the container 402. The generally cylindrical body has an outer surface 422c and is closed by a septum 422d at a first end, and open at a second end. A flange 422b extends from the generally cylindrical body 422a at the first end.

The stopper 422 is held in place by an end cap 424. The end cap 424 can be a crimped seal, a snap-fit cap, or other cap assembly configured to hold the stopper 422 in place within a neck of a container 402.

The stopper assembly 420 of FIG. 8 differs from the stopper assembly of FIG. 7 in the placement of the resilient sealing portion 410. As shown in FIG. 8, an abutment surface of the flange 422b, configured to abut the rim of the container 402, comprises a groove 412. The resilient sealing portion 410 is at least partially seated within the groove 412 and is configured to form a seal with the rim of the container.

The resilient sealing portion 410 can comprise an O-ring or a spring energised seal, similar to the resilient portions 110, 110′, 110″ and 310 described above. In this configuration, by providing an O-ring or spring energised seal comprising a core material and (at least a partial) outer jacket, the core material can be chosen to minimise thermal expansion and/or contraction of the resilient portion(s), whilst the jacket material can be chosen to form a suitable seal with a sealing surface of a syringe barrel or vial flange.

The configuration of the resilient sealing portion(s) 310, 410 described above is such that a temperature resilient seal may be formed between the stopper assembly and the container so that the container assembly is operable to contain a medicament between the seal and the opening at: (a) room temperature (e.g. approximately 20° C.), and (b) during freeze storage of the medicament, with the plunger assembly still being movable relative to the container at room temperature.

Referring again to FIGS. 7 and 8, the stopper assemblies 320, 420 are each shown to comprise one groove, configured to receive a resilient sealing portion. However, it will be appreciated that two or more grooves with resilient portions may be provided.

Moreover, although the stopper assemblies of FIGS. 7 and 8 are described as suitable for use in the container system shown in FIG. 2, it will be appreciated that the stopper assemblies 320, 420 described above may be used to seal a conventional vial (e.g., having a single opening to be sealed by a stopper).

Operation of the container assembly described with reference to FIG. 5 will now be described in more detail with reference to FIGS. 9 to 12.

As shown in FIG. 9, container sub assembly 201 is prepared for filling with plunger 206 inserted into container 202, and insert assembly 230 is secured in place over the second end of the container 202. The first end of the container 202, comprising the opening 204, is open (i.e., not closed by stopper assembly 220). Medicament M is introduced into the container 202 via the first opening 204. The stopper assembly 206 prevents egress of the medicament M from the second end of the container.

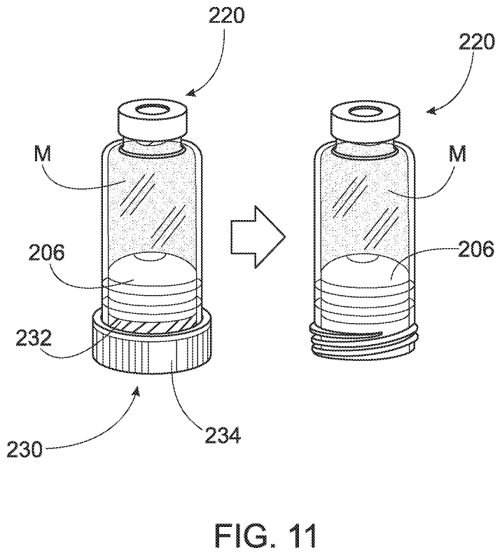

Turning to FIG. 10, stopper assembly 220 is applied to the container 202 to close the open end 204 of the container 202. The stopper is placed within the neck of the container to form a seal preventing egress of medicament, and the first end cap is used to secure the stopper in place. The container assembly is now a sealed enclosure containing medicament M.

FIG. 11 shows the filled container assembly of FIG. 10 being prepared ready for use in dispensing a dose of medicament. As shown in FIG. 11, the second end cap 234 is removed from the container 202 (e.g., by unscrewing if it is configured for threaded engagement with the container 202) and the insert 322 removed. With the end cap 234 and insert 232 removed, it is possible to access the plunger assembly 206 through the second open end of the container 202. At this stage, the plunger assembly 206 can be coupled to a drive element such as plunger rod (not shown in FIG. 11) under the influence of which the plunger assembly may be driven forwards along the container body and the first end of the container 202 can be coupled to a fluid delivery conduit to allow medicament M to be discharged from the container 202 under the influence of an advancing plunger assembly 206.

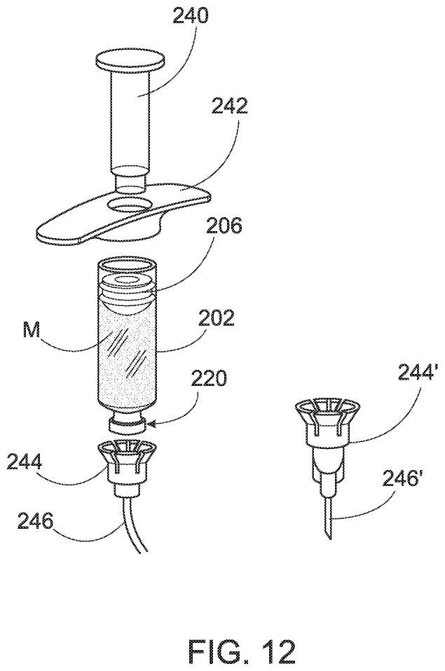

FIG. 12 shows an example configuration in which the container assembly of FIG. 11 can be deployed. As shown in FIG. 12, a drive element in the form of plunger rod 240 can be configured for coupling to the plunger assembly 206. The coupling may be by push-fit engagement between the plunger assembly 206 and the plunger rod 240, by threaded engagement, or by other means. A flange extension 242 can be affixed at or near the second end of the container 202 to provide a bearing surface for a user's fingers as they depress the plunger rod 240.

To allow medicament M to be delivered from the container 202, the first end of the container 202 is configured to be coupled to a delivery conduit. The delivery conduit may take the form of a delivery conduit hub 244 and flexible delivery conduit 246 configured to deliver medicament to a patient via e.g., an infusion set. Alternatively, the delivery conduit may take the form of a delivery conduit hub 244′ and a needle 246′ via which a medicament M can be injected into a patient.

In either case, the delivery conduit hub 244, 244′ can be configured to pierce a septum of the stopper assembly 220 to establish fluid communication between the delivery conduit (e.g., tube 246, or needle 246′) and the interior volume of the container 202 containing medicament M.

Although FIG. 12 shows a system in which the container assembly of FIG. 5 is combined with additional features designed to facilitate manual discharge of medicament M from container 202, it will be appreciated that the container assemblies described herein may also be used in combination with automatically driven plunger assemblies. For example, the container assembly may be configured for use in an automatic injection device comprising an electromechanical drive assembly. One of the advantages of providing a container assembly comprising a plunger assembly and a removable stopper and/or end cap is that the container assemblies may be used in a variety of contexts (e.g. inserted into an automatically driven injection device, combined with accessories to allow manual injection of the medicament, or used similar to a vial from which the medicament may be extracted through the septum of the stopper by using a separate hypodermic syringe). Moreover, the design and shape of the container assemblies described herein may facilitate storage and freezing of the container assemblies because the shape can be accommodated in cooling and storage boxes currently used for storage of cell and gene therapies (e.g., mechanical freezers, liquid nitrogen Dewars, etc.).

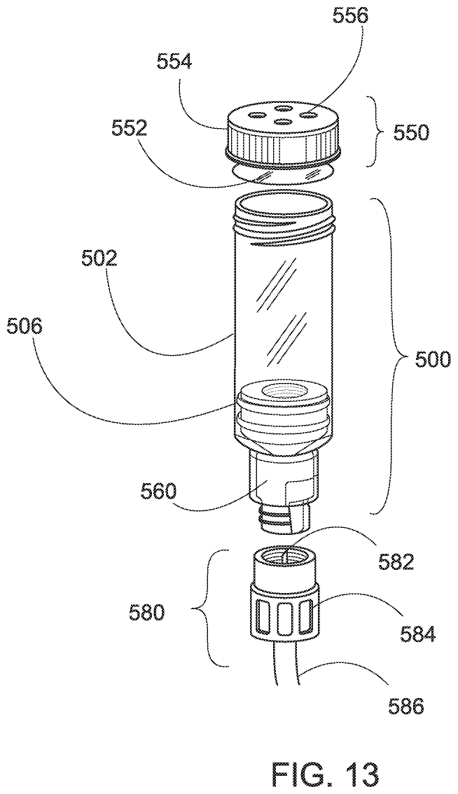

Turning now to FIG. 13, a closed fill container system according to the disclosure will now be described in more detail. As shown in FIG. 13, the container system comprises a container assembly 500, which is equivalent in structure to the container assembly 100 described with reference to FIGS. 1 to 4. The container assembly 500 comprises a container 502 and a plunger assembly 506 disposed therein. The plunger assembly 506 has a similar construction to the plunger assembly 102 described with reference to FIGS. 1 to 4.

As shown in FIG. 13, the container assembly is closed at a second end (top end in FIG. 13) by a vented cap assembly 550. The vented cap assembly 550 comprises a vented end cap 554 and a filter 552.

The vented cap 554 comprises at least one, and optionally two or more openings 556 configured to allow air to pass into (and out of) of the container 502 through the opening(s) 556. A filter 552 extends across the opening(s) 556 to prevent the ingress of particulates into the container 502. The filter 552 may be, for example, a 0.22 μm filter.

The vented cap assembly 550 shown in FIG. 13 is configured to be secured in place over the second opening (at the second end of the container 502) with a threaded engagement between the vented end cap 554 and the container 502. However, the skilled person will appreciate that other engagement means between the end cap assembly 550 and the container 502 may be provided, such as a push- or snap-fit connection, a bayonet connection, etc. Moreover, frangible connections between the vented end cap and the container may also be used to help ensure that the container is used only once. One of the advantages of providing a vented cap as described above is that any headspace (air behind the plunger assembly) can be vented through the openings in the cap.

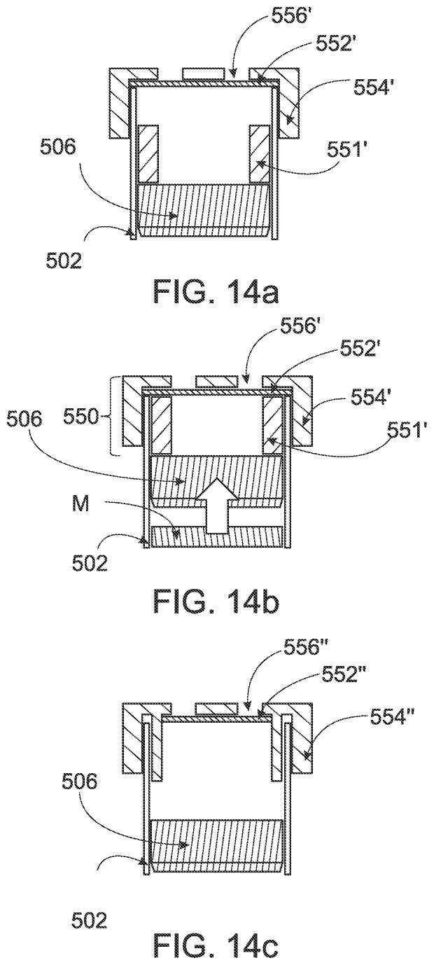

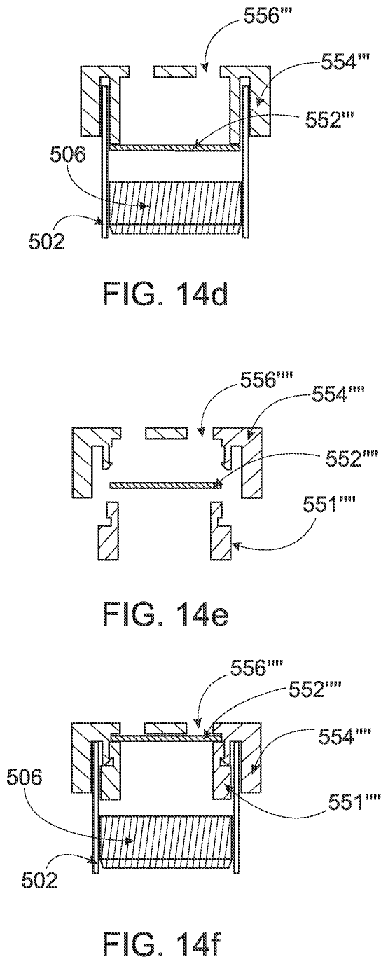

As will now be described with reference to FIGS. 14a-14f, the vented cap assembly 550 can take different forms.

For example, in a first arrangement (shown in FIG. 14a) the vented cap assembly can comprise a vented end cap 554′ and a filter 552′ similar to the vented cap assembly 550 illustrated in FIG. 13. The vented end cap 554′ comprises openings 556′, similar to openings 556 of assembly 550 shown in FIG. 13. Additionally, the vented cap assembly of FIG. 14a comprises an insert 551′, The insert 551′ is configured to be disposed between the open end of the container 502 (closed by the vented end cap 554′) and the plunger 506. In this way, the insert 551′, confined between the end cap 554′ and the plunger 506 determines a minimum distance between the open end of the container 502 and the plunger 506. By defining the position of the plunger 506 in this way, the volume of medicament M in the container 506 can be controlled.

FIG. 14a shows the end cap 554′, insert 551′, and plunger 506 of a container assembly according to the disclosure before filling. As shown in FIG. 14a, the insert 551′ is confined between the plunger 506 and the end cap 554′, which is secured in place over an open end of the container 502. In this initial position, the container 502 has not yet been (completely) filled with medicament M, and so the insert 551′ is free to move between the end cap 554′ and the plunger 506.

Turning to FIG. 14b, and the container 502 is filled with medicament M (via the check valve, shown in FIG. 13), the plunger 506 is pushed towards the end cap 554′ as the medicament M fills the internal volume of the container 502. The plunger 506 is free to move towards the end cap 554′ until such a point as the insert 551′ abuts both the plunger 506 and the end cap 554′ and will not allow the plunger 506 to advance further. In this way, a minimum distance between the open end of the container 502 and the plunger 506 can be defined, to control the maximum fillable internal volume of the container 502.

FIGS. 14c and 14d show two further arrangements for the vented cap assembly in which the spacing insert and the end cap care combined into a single monolithic component (or are otherwise fixedly secured to each other).

As shown in FIG. 14c, the vented end cap assembly comprises an end cap portion 554″ and an insert portion 551″ integrally formed therewith. The end cap portion 554″ is configured to engage the container 502, whilst the insert portion 551″ is configured to extend from the end cap portion 554″, within the interior volume of the container 502, to define a minimum distance between the open end of the container 502 and the plunger 506. The insert portion 551″ can comprise an extension (e.g., a generally cylindrical extension) or a plurality of extensions (e.g., a plurality of legs or posts) extending from the end cap portion 554″ towards the plunger 506. The extension(s) are configured to provide an abutment surface against which the plunger 506 bears to prevent further advancement of the plunger 506 towards the open end of the container 502. In the arrangement shown in FIG. 14c, the filter 552″ sits against the end cap portion 554″, adjacent the openings 556″. The arrangement of FIG. 14d is similar to the arrangement of FIG. 14c, except that the filter 556′″ is spaced apart from the openings 556′″, by the insert portion 551′″.

Turning now to FIGS. 14e and 14f, yet another arrangement for a vented end cap assembly is shown. FIG. 14e shows the arrangement in a disassembled state, in which the insert and the end cap are disconnected from each other. FIG. 14f shows the arrangement in an assembled state, in which the end cap and the insert are connected.

In the arrangement shown in FIGS. 14e and 14f, the vented end cap assembly can comprise a vented end cap 554″″ and insert 551 ″″ configured for selective engagement with each other. The engagement between the end cap 554″″ and the insert 551″″ can be snap-fit, interference fit, or a threaded engagement. In this configuration, the end cap 554″″ is configured to engage the container 502, whilst the insert 551″″ is configured to define a minimum distance between the open end of the container 502 and the plunger 506. In such an arrangement, because the end cap 554″″ and the insert 551 ″″ are connected to each other (e.g., by snap fit engagement) removal of the end cap 554″″ from the container 502 also removes the insert 551 ″″ from the open end of the container 502, leaving the plunger 506 accessible for use. It will be appreciated that an advantage of this connected but modular construction of the insert 551 ″″ and the end cap 554″″ means that the size of the insert 551 ″″ can be varied (to vary the minimum distance between the plunger and the open end of the container) independently of the end cap 554″″, but removal of the end cap 554″″ can still remove the insert 551 ′″ from the open end of the container 502. Another advantage of such an arrangement is that the filter 552″″ can be captured or otherwise confined between the end cap 554″″ and the insert 551″″.

In an alternative embodiment, the vented cap assembly 550 may further comprise a hollow insert, similar to the previously described insert 232, except that the hollow insert of the vented cap assembly 550 would be hollow and have two open ends to allow air to pass through the hollow insert. In another embodiment, the hollow insert and the vented cap 554 may be monolithic (formed as a single piece), so that removing the vented cap 554 would simultaneously remove the hollow insert. In such an embodiment, the filter 552 would be embedded within the monolithic component. In a preferred embodiment, the hollow insert and vented cap 554 may be provided as separate components, but provided with a coupling, such as a snap-fit, so that the two components may be attached to capture the filter between the two components.

The vented end cap and/or hollow insert may be formed from one or more rigid polymeric materials, such as polyethylene (PE), polypropylene (PP), polyvinyl chloride (PVC), polystyrene (PS), polyethylene terephthalate (PET) or acrylonitrile butadiene styrene (ABS). Advantageously, the material chosen for the vented end cap is compatible with cryopreservation media such as dimethyl sulfoxide (DMSO).

The filter may be formed from paper, cellulose, nylon, polycarbonate, polyvinylidene fluoride, polypropylene, polyethersulfone, polytetrafluoroethylene, mixed cellulose esters, and others.

A check valve 560 is provided at the first end of the container 502 and is configured to allow the flow of fluid into the container 502. The check valve can be a one-way bicuspid valve, but other types of one-way valve can also be used. In at least some configurations, the valve 560 comprises silicone.

The valve 560 can be co-moulded with the container 502. A co-moulded valve can be particularly advantageous for continuity of sterility and ease-of-use, but it will be appreciated that the valve 560 may also be formed separately and disposed within the container 502. The check valve may be formed of the same material as the body of the container. Alternatively, the check valve can be a separate sub-assembly made from one or more rigid polymeric materials, such as polyethylene (PE), polypropylene (PP), polyvinyl chloride (PVC), polystyrene (PS), polyethylene terephthalate (PET) or acrylonitrile butadiene styrene (ABS). Advantageously, the material chosen for the check valve is compatible with cryopreservation media such as dimethyl sulfoxide (DMSO).

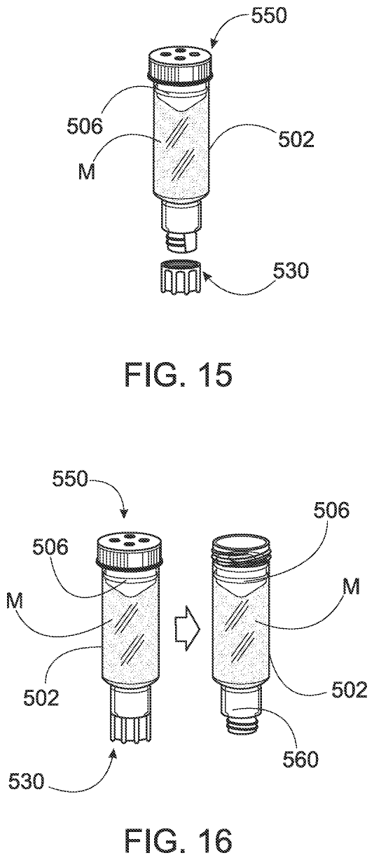

In some configurations, a check valve can be configured to operate in the opposite flow direction to the check valve 560 (e.g., to allow flow out of the container 502) when engaged with a complementary vial adapter. A possible vial adapter configuration will be described in more detail with reference to FIGS. 15 to 17.

The container assembly 500 can be supplied with a valve adapter 580. The valve adapter 580 can comprise a housing 582 configured to engage the first end of the container 502. The housing 584 comprises a valve engagement portion 582, for example a needle or probe, which can be inserted into the valve 560 and is configured to allow flow through the valve 560 in a direction out of the container 502 (i.e., in a direction opposite the direction of flow allowed through the valve 560 without the adapter in place). The valve engagement portion 582 is configured to provide fluid communication between the valve 560 and a conduit 586. Via the valve adapter 580, medicament can therefore be discharged from the container 502 through the valve 560 located at the first end. This configuration may be advantageous because it can allow easier filling of the container, with the one-way valve preventing the leakage of fluid from the container once filled.

In the event that the check valve 560 is configured to allow one-way flow out of the container 502, the valve adaptor 580 can be configured to open the valve 560 to allow the flow of fluid through the valve 560 into the container via the valve adaptor 580.

Filling of the container 502 will now be described in more detail with reference to FIGS. 15 and 16.

As shown in FIG. 13 (described in detail above), in an initial configuration, the plunger assembly 506 is located towards the first end of the container 502, adjacent the check valve 560. The vented cap assembly 550, is secured in place over the second open end of the container 502.

To allow the container 502 to be filled, the valve adapter 580 is secured to the first end of the container 502, such that the valve engagement portion 582 establishes fluid communication between the conduit 586 and the internal volume of the container 502. In this way, medicament can be introduced into the container 502 through the valve adaptor 580.

Turning now to FIG. 15, as medicament M is introduced into the container 502 through the valve adaptor 580, the plunger assembly is driven along the length of the container 502 toward the second end (i.e., toward the vented end cap assembly 550). When the container 502 is filled with medicament M, the plunger assembly 506 will be at the second end of the container 502, as shown in FIG. 15. With the container 502 full (or filled to the desired level), a valve cover 530 can be placed over the first end of the container 502. The valve cover 530 may be configured for threaded engagement with the container 502, or another fastening means may be used. For example, the valve cover 530 may be push fit or snap fit onto the container, or a bayonet fastening may be used. Moreover, frangible connections between the valve cover and the container may also be used to help ensure that the container is used only once.

FIG. 16 shows the container assembly of FIG. 13 being prepared ready for use in dispensing a dose of medicament. As shown in FIG. 16, the vented end cap assembly 550 is removed from the second end of the container 502. This allows access to the plunger assembly 506 through the second open end of the container 502. This can allow, for example, a drive element such as a plunger rod (not shown) to be connected to the plunger assembly 506 to allow the plunger assembly to be advanced to deliver a dose of medicament M. The plunger assembly 506 may also be configured to mate with a drive element of an automatic injection device.

The valve cover 530 is also removed, from the first end of the container. Removal of the valve cover 530 allows access to the check valve 560. Although not shown in the drawings, it will be appreciated that the first end of the container 502 can be configured to mate with a hub that provides fluid communication to a delivery conduit. The delivery conduit can take the form of a needle or a flexible conduit through which medicament M can be delivered. The check valve 560 can ensure that medicament M can exit the container 502 via the check valve 560, but that the container 502 cannot be refilled without a valve adaptor 580.

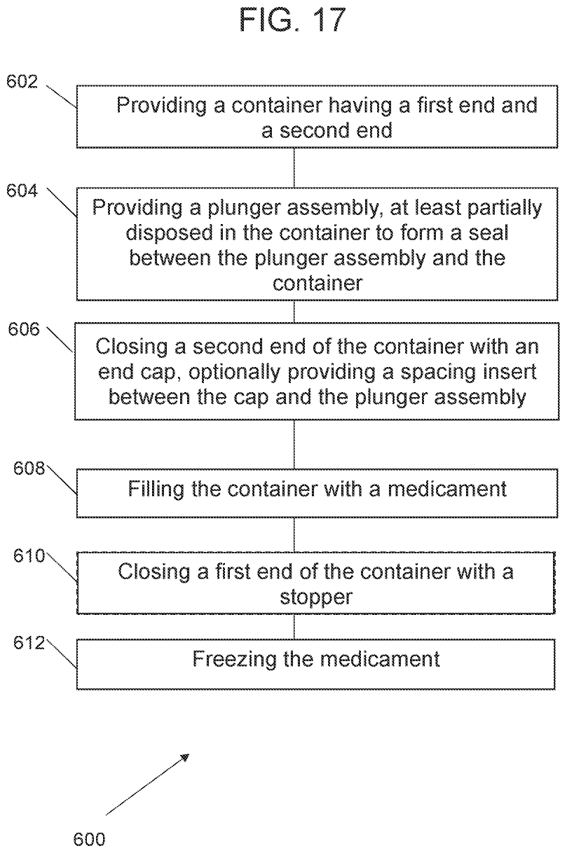

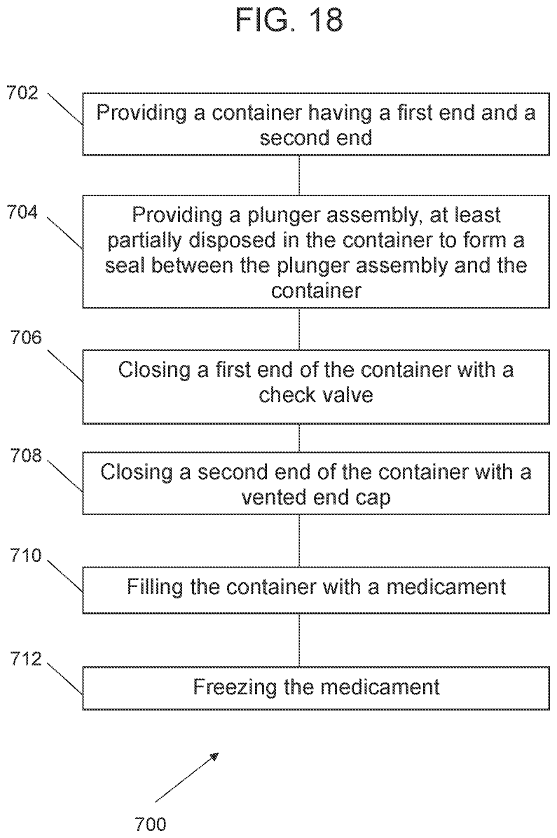

Turning now to FIGS. 17 and 18, exemplary methods of manufacturing and/or filling a container assembly according to the disclosure will now be described. It will be appreciated that the disclosure includes methods of manufacturing an unfilled container, methods of manufacturing a filled container, and/or methods of filling a pre-manufactured container.

In general terms, methods according to the disclosure include a method of filling a container assembly according to any of the configurations described above. For example, the container assembly can comprise (i) a container having a first end and a second end, the first end comprising a first opening; and (ii) a plunger assembly at least partially disposed in the container. The plunger assembly can comprise: (i) a main body; and (ii) at least one resilient portion at least partially retained by a groove in the main body and forming a seal with the container. The plunger assembly is configured to form a temperature resilient seal with the container such that the container assembly is operable to contain a medicament between the seal and the first opening at: (a) room temperature, and (b) during freeze storage of the medicament; and be moveable relative to the container at room temperature. The method of filling the container described above includes the step of filling the container assembly with the medicament via the opening in the first end. After filing the container assembly with the medicament, an optional step of freezing the medicament contained in the container assembly may also be performed. The freezing step can comprise cooling the contents of the container to between −50° and −200° C. This method can further comprise the step of sterilising the container assembly, before or after filling the container.

In addition to a method of a container assembly to provide a pre-filled assembly, the present disclosure also provides methods of forming (and optionally filling) the containers described above.

FIG. 17 shows, in schematic form, a method 600 of forming and filling a container assembly with a medicament. As shown in FIG. 17, at a first step 602, a container is provided having a first end and a second end. At a second step 604, a plunger assembly is disposed at least partially in the container, to form a seal between the plunger assembly and an inner wall of the container. The plunger assembly may be configured according to any of the embodiments described above with reference to FIGS. 1 to 16 and is configured to maintain a seal between the plunger assembly and the container at very low temperatures (e.g., less than −50° C.) and to allow sliding movement of the plunger assembly relative to the container at room temperature (e.g., at around 20° C.).

At step 606, the second end of the container is closed with an end cap. A spacing insert is optionally provided between the plunger assembly and the end cap to maintain a pre-defined minimum distance between the second end of the container and the plunger assembly.

At step 608, the container is filled with a medicament. Note that the order of steps 606 and 608 may be reversed so that the container is filled with medicament before the end cap is positioned to close the second end of the container. The medicament M is introduced into the space defined between the plunger assembly and the first end of the container.

At step 610, after the filling step, the first end of the container is closed with a stopper. A first end cap may be positioned over the stopper to maintain the stopper in place over the first end of the container. Once the first end of the container is closed with the stopper, the medicament M is sealed within the container.

Optionally, at step 612, the medicament is frozen. The medicament may be frozen by reducing its temperature to below 0° C., optionally between −50° C. and −200° C. This method can further comprise the step of sterilising the container assembly, before or after filling the container.

FIG. 18 shows, in schematic form, another method 700 of forming (and optionally filling) a container assembly with a medicament. As shown in FIG. 18, at a first step 702, a container is provided having a first end and a second end. At a second step 704, a plunger assembly is disposed at least partially in the container, to form a seal between the plunger assembly and an inner wall of the container. The plunger assembly may be configured according to any of the embodiments described above with reference to FIGS. 1 to 16 and is configured to maintain a seal between the plunger assembly and the container at very low temperatures (e.g., less than −50° C.) and to allow sliding movement of the plunger assembly relative to the container at room temperature (e.g., at around 20° C.).

At step 706, the first end of the container is closed with a check valve. At step 708, the second end of the container is closed with a vented end cap. A filter may be provided between the vented end cap and the second end of the container during this step.

The order of steps 706 and 708 may be reversed so that the vented end cap is positioned before the check valve. Moreover, in at least some embodiments, the check valve is co-moulded with the container so steps 702 and 706 happen substantially simultaneously.

The method of manufacturing a container assembly may end after step 708, and the container assembly may be provided to a user in an empty state. However, the method may also comprise optional filling and/or freezing steps, as will be described below.

At step 710, the container is filled with a medicament. The medicament may be introduced into the container through the check valve. A valve adaptor may or may not be used for this step, depending on the permitted flow direction of the check valve. The filling step drives the plunger assembly from its initial position at the first end of the container (adjacent the check valve) towards the second end of the container. In this way, the medicament M is introduced into a space defined between the plunger assembly and the first end of the container.

After step 710, the medicament is optionally frozen. The medicament may be frozen by reducing its temperature to below 0° C., and optionally between −50° C. and −200° C. This method can further comprise the step of sterilising the container assembly, before or after filling the container.

Although, the present disclosure describes plunger assemblies configured to maintain a seal at very low temperatures in the context of exemplary container assemblies, it will be appreciated that the present disclosure also provides plunger assemblies in isolation from the wider container assembly.

In one aspect of the disclosure, a plunger assembly comprises a main body; and at least one resilient portion at least partially retained by a groove in the main body. The resilient portion is configured to form a seal with a container for containing a medicament. The plunger assembly is further configured to: form a temperature resilient seal with a container such that the container assembly is operable to contain a medicament between the seal and a first opening of the container at: (a) room temperature, and (b) during freeze storage of the medicament; and to allow movement of the plunger assembly relative to the container at room temperature.

The plunger assembly can take the form of the plunger assemblies described with reference to FIGS. 2a to 4.

Similarly, although the present disclosure describes stopper assemblies configured to maintain a seal at very low temperatures in the context of exemplary container assemblies, it will be appreciated that the present disclosure also provides stopper assemblies in isolation from the wider container assembly.

In one aspect of the disclosure, a stopper assembly is provided that is configured to seal an opening of a container. The stopper assembly comprises a stopper configured to be at least partially disposed in the opening of a container for containing a medicament. The stopper assembly also comprises at least one resilient sealing portion at least partially retained by the stopper and configured to form a seal with the container.

The stopper can comprise a generally cylindrical body having an outer surface comprising at least one groove, and wherein the at least one resilient sealing portion is at least partially retained by the at least one groove.

The stopper assembly can take the form of the assemblies described with reference to FIGS. 7 and 8.

The preceding detailed description describes container assemblies, and associated systems and methods that are configured to maintain a seal at cryogenic temperatures, and also allow movement of a plunger within the system at room temperature. However, the skilled person will understand that the invention is not limited to use in connection with the exemplary device described here. Rather, one or more benefits associated with the present invention may be implemented in connection with other drug delivery systems, as will be apparent to the skilled person in light of the preceding detailed description.

It will also be understood that, where used, the terms “proximal”, “distal”, “front”, “back”, “side”, “top” and “bottom” are used for convenience in interpreting the drawings and are not to be construed as limiting. The term “comprising” should be interpreted as meaning “including but not limited to”, such that it does not exclude the presence of features not listed.

The embodiments described and shown in the accompanying drawings above are provided as examples of ways in which the invention may be put into effect and are not intended to be limiting on the scope of the invention. Modifications may be made, and elements may be replaced with functionally and structurally equivalent parts, and features of different embodiments may be combined without departing from the disclosure.

1. A container assembly for containing and delivering a medicament, the container assembly comprising:
a container having a first end and a second end, the first end comprising a first opening; and
a plunger assembly at least partially disposed in the container, the plunger assembly comprising:
a main body; and
at least one resilient portion at least partially retained by a groove in the main body and forming a seal with the container, wherein the plunger assembly is configured to:
form a temperature resilient seal with the container such that the container assembly is operable to:
contain a medicament between the seal and the first opening at: (a) room-temperature and (b) during freeze storage of the medicament; and
be moveable relative to the container at room temperature.

2. The container assembly of claim 1, wherein the plunger assembly is configured to be moved relative to the container by a drive element.

3. The container assembly of claim 1, wherein the resilient portion is configured to provide an interference fit with the container.

4. The container assembly of claim 1, wherein the at least one resilient portion comprises a core and a jacket, the jacket at least partially encapsulating the core.

5. The container assembly of claim 4, wherein the jacket comprises a fluoropolymer material.

6. The container assembly of claim 1, wherein the at least one resilient portion is an encapsulated O-ring.

7. The container assembly of claim 1, wherein the at least one resilient portion comprises a spring energised seal, and wherein the spring energised seal comprises a spring and a sealing ring.

8. The container assembly of claim 7, wherein the spring is a continuous contact spring, a cantilever spring, a helical-wound spring, or an elliptical coil spring.

9. The container assembly of claim 7, wherein the sealing ring comprises a fluoropolymer material.

10. The container assembly of claim 1, wherein the plunger assembly comprises at least two resilient portions, and wherein:
one of the at least two resilient portions is configured to form the temperature resilient seal; and
the other of the at least two resilient portions is configured to stabilize the plunger assembly in the container.

11. The container assembly of claim 1, wherein room temperature is around 20° C. and the freeze storage of the medicament is carried out at −50° C. to −200° C.

12. The container assembly of claim 1, wherein freeze storage of the medicament is carried out at cryogenic or at ultralow temperatures.

13. The container assembly of claim 1, further comprising a stopper assembly configured to seal the first opening, the stopper assembly comprising:
a stopper at least partially disposed in the opening of the container; and
at least one resilient sealing portion at least partially retained by the stopper and forming a seal with the container.

14. The container assembly of claim 13, further comprising a first end cap configured to maintain the stopper in position at least partially within the first opening of the container.

15. The container assembly of claim 1, further comprising an insert at least partially disposed in the container, the insert being configured to maintain a minimum distance between the plunger assembly and the second end of the container.

16. The container assembly of claim 15, further comprising a second end cap configured to engage the container and retain the insert in the container.

17. The container assembly of claim 1, further comprising a vented end cap configured to engage the container at or near the second end of the container, and wherein:
the second end comprises a second opening; and
the vented end cap comprises one or more openings configured to allow air to pass into or out of the container through the second opening.

18. The container assembly of claim 1, further comprising a one-way check valve at least partially disposed at or near the first end of the container, the one-way check valve configured to allow fluid flow into the container via the first opening.

19. The container assembly of claim 18, further comprising a valve cover configured to engage the container at or near the first end of the container to protect the one-way check valve.

20. The container assembly of claim 18, further comprising a valve adaptor configured to engage the container at or near the first end of the container, the valve adapter configured to open the one-way check valve and allow fluid flow out of the container.
